(19) United States
(12) Patent Application Publication
(10) Pub. No.: US 2006/0205056 A1
(43) Pub. Date: Sep. 14, 2006

(54) Enzymatic method of making ethyl 3-hydroxy-3-phenylpropionate and their esters

(76) Inventors: Soon Ook Hwang, Daejeon (KR); Hye Youn Ryu, Deajeon (KR)

Correspondence Address: IPLA P.A., 3580 WILSHIRE BLVD., 17TH FLOOR, LOS ANGELES, CA, 90010, US

(21) Appl. No.: 10/544,032

(22) PCT Filed: Feb. 5, 2004
(86) PCT No.: PCT/KR04/00211

§ 371 Date: Aug. 1, 2005

(30) Foreign Application Priority Data
Feb. 6, 2003 (KR) 10-2003 0007420

(51) Int. Cl. C12P 7/52; C12P 41/00

(52) U.S. Cl. 435/280; 435/141

(57) ABSTRACT

The present invention provides a new process for the preparation of optically active ethyl 3-hydroxy-3-phenylpropionate and their esters by enzymatic method. More particularly, the racemic ethyl 3-hydroxy-3-phenylpropionate is converted to optically active ethyl 3-hydroxy-3-phenylpropionate and their esters by enzymatic reaction with acylating agent. This invention may be employed for the synthesis of individual enantiomers of ethyl 3-hydroxy-3-phenylpropionate.

### TECHNICAL FIELD

The present invention relates to a new process for the preparation of optically active ethyl 3-hydroxy-3-phenylpropionate and its esters by enzymatic method. More particularly, the present invention relates to a process for the production of optically active ethyl 3-hydroxy-3-phenylpropionate and esters using lipases with acyl donors.

Optically active (R)- and (S)-ethyl 3-hydroxy-3-phenylpropionate are important intermediates in the synthesis of antidepressants such as Fluoxetine, Tomoxetine and Nisoxetine.

(S)-ethyl 3-hydroxy-3-phenylpropionate produced by the process of this invention is more useful than (S)-3-hydroxy-3-phenylpropanenitrile or (S)-3-chloro-1-phenyl-1-propanol in the synthesis of Fluoxetine. Because (S)-ethyl 3-hydroxy-3-phenylpropionate can be easily converted to an intermediate for the synthesis of Fluoxetine.

### BACKGROUND ART

Conventional methods relating to the preparation of the chiral intermediate of Fluoxetine are as follows;

There is the biological method for preparing (S)-ethyl 3-hydroxy-3-phenylpropionate using microorganisms. Kumar et al. produced (S)-ethyl 3-hydroxy-3-phenylpropionate (85ee %) by the reduction of ethyl benzoylacetate using bakers' yeast (see Tetrahedron Letters, 32(16), 1901-1904(1991)) and Chenevert et al. obtained (S)-ethyl 3-hydroxy-3-phenylpropionate (98ee %) by the reduction of ethyl benzoylacetate using Geotrichum candidum (see Tetrahedron, 48(33), 6769-6776 (1992)).

On the other hand, the lipase-mediated methods are as follows. (R)-3-chloro-1-phenyl-1-propanol (97.3ee %) and (S)-3-chloro-1-phenyl-1-propanylchloroacetate were obtained by the hydrolysis of 3-chloro-1-phenyl-1-propanylchloroacetate using lipase SAM II. (see Tetrahedron:Asymmetry, 3(4), 525-528(1992))

Also Garcia et al. obtained (S)-amide (66ee % at 11% conversion) by the aminolysis of racemic ethyl 3-hydroxy-3-phenylpropionate with benzylamine using lipase CAI. However it is not easy to use this process for the production of Fluoxetine, because it takes so much time and the optical purity is too low.

According to Raju et al., (R)- and (S)-3-chloro-1-phenyl-1-propanol were prepared from racemic 3-chloro-1-phenyl-1-propanol using lipase PS with isopropenyl acetate as the acylating agent in heptane. (S)-3-chloro-1-phenyl-1-propanol (99ee % at 52% conversion) and (R)-ester (92.4ee % at 49% conversion) were obtained after 96 hr of reaction time. (see Tetrahedron:Asymmetry, 6(7), 1519-1520(1995))

(S)-3-hyrodxy-3-phenyl-3-propanenitrile (99ee % at 46% conversion) and (R)-3-acetoxy-3-phenyl-3-propanenitrile were obtained by the transesterificatio of racemic 3-hyrodxy-3-phenyl-3-propanenitrile which was synthesized from styrene oxide, with vinyl acetate as the acylating agent using lipase PS-C. (see Tetrahedron:Asymmetry, 13, 2039-2051(2002))

### DISCLOSURE OF INVENTION

As mentioned above, some intermediates used in the synthesis of antidepressants such as Fluoxetine, Tomoxetine or Nisoxetine are 3-hyrodxy-3-phenyl-3-propanenitrile, 3-chloro-1-phenyl-1-propanol and ethyl 3-hydroxy-3-phenylpropionate. Particularly, chiral 3-chloro-1-phenyl-1-propanol or chiral 3-hyrodxy-3-phenyl-3-propanenitrile was prepared by an enzymatic resolution process using lipases. Chiral ethyl 3-hydroxy-3-phenylpropionate was obtained by microbiological reduction of ethyl benzoylacetate by bakers' yeast.

As the result of the intensive studies associated with the process of preparing a chiral intermediate, ethyl 3-hydroxy-3-phenylpropionate, the invention herein was devised based on the fact that racemic ethyl 3-hydroxy-3-phenylpropionate can be synthesized by the reduction of commercially avaiable ethyl benzoylacetate or from other intermediates such as 3-hydroxy-3-phenylpropanenitrile, and lipase-mediated esterification of ethyl 3-hydroxy-3-phenylpropionate has not been reported until now.

This invention is accomplished based on the fact that kinetic resolution of ethyl 3-hydroxy-3-phenylpropionate via lipase-catalyzed transesterification with acylating agent gives the (S)-ethyl 3-hydroxy-3-phenylpropionate and the corresponding (R)-esters.

Therefore, the objective of this invention is to provide the process for preparing optically pure (S)-ethyl 3-hydroxy-3-phenylpropionate and the corresponding (R)-esters which can be converted to (R)-ethyl 3-hydroxy-3-phenylpropionate, from racemic ethyl 3-hydroxy-3-phenylpropionate with acylating agent using lipases.

This invention relates to the process for preparing optically pure (S)-ethyl 3-hydroxy-3-phenylpropionate and the corresponding (R)-esters from racemic ethyl 3-hydroxy-3-phenylpropionate via lipase-catalyzed transesterification with acylating agent in organic solvent or with acylating agent only without using organic solvent.

This invention is explained in more detail as follows.

Lipases used in the present invention include those at powder or immobilized lipase. For the lipase, commercially available ones and, if necessary, home-made ones can be used. Non-limiting examples of the commercially available lipase include Novozyme 435 from Novo company, those manufactured by Amano company such as lipase PS, PS-C and PS-D and CPL (Candida rugosa lipase) from Sigma company.

For the reaction in this invention, organic solvents include isopropylether, t-butylmethylether, tetrahydrofuran and methylenechloride and so on. And acylating agents which can be also used as organic solvent include vinyl acetate, vinyl propionate, isopropenylacetate, acetic anhydride and butyric anhydride and so on.

Meanwhile, the racemic ethyl 3-hydroxy-3-phenylpropionate and both enantiomers, (R)- and (S)-ethyl 3-hydroxy-3-phenylpropionate were determined by a gas chromatography (Donam Instruments Inc. Model DS 6200). Analysis conditions are as follows. Conversion of the reaction was determined by a gas chromatography (Donam Instruments Inc. Model DS 6200) with a flame ionization detector and a BP-1 capillary column (0.53 mm×30 m, SGE) using Helium as the carrier gas. The oven temperature was maintained initially at 70° C. for 5 min and then raised at the rate of 10° C./min to 220° C., and maintained for 15 minutes. The typical retention time of the components in this invention was:
(±)-ethyl 3-hydroxy-3-phenylpropionate-25.56 min
(±)-ethyl 3-O-acetyl-3-phenylpropionate-23.8 min
(±)-ethyl 3-O-propionyl-3-phenylpropionate-24.6 min
(±)-ethyl 3-O-butanoil-3-phenylpropionate-25.8 min

Its enantiomeric excess was determined by a gas chromatography (Donam Instruments Inc. Model DS 6200) with a flame ionization detector and a chiral column G-TA (0.32 mm×30 m, Astech) using Helium as the carrier gas. The oven temperature was maintained initially at 120° C. for 30 min, and then raised at the rate of 30° C./min to 170° C., and maintained for 15 min, and Column head pressure was maintained at 4 psi. Typical retention time was:
(R)-ethyl 3-hydroxy-3-phenylpropionate-37.3 min
(S)-ethyl 3-hydroxy-3-phenylpropionate-37.5 min
(R)-ethyl 3-O-acetyl-3-phenylpropionate-37.88 min
(S)-ethyl 3-O-acetyl-3-phenylpropionate-38.12 min
(R)-ethyl 3-O-propionyl-3-phenylpropionate-41.34 min
(S)-ethyl 3-O-propionyl-3-phenylpropionate-41.65 min
(R)-ethyl 3-O-butanoyl-3-phenylpropionate-46.46 min
(S)-ethyl 3-O-butanoyl-3-phenylpropionate-46.97 min

A better understanding of the present invention may be obtained through the following examples which are set forth to illustrate, but are not to be construed as the limit of the present invention.

### EXAMPLE 1

Vinyl acetate (0.2 ml, 4% v/v) and t-butymethylether (4.75 ml) were placed in a 15 ml vial. Then, ethyl 3-hydroxy-3-phenylpropionate (0.05 ml, 1% v/v) and PS-C (0.2 g, 4% w/v) were added to the mixture. The reaction mixture was shaking at 150 rpm and 45° C. The supernatant of the reaction mixture was withdrawn after 20 hours and its components were determined by a gas chromatography (Donam Instruments Inc. Model DS 6200) as mentioned above. The results are 100% ee for (S)— ethyl 3-hydroxy-3-phenylpropionate at 55.6% of conversion and 97.8% ee for corresponding (R)-ethyl 3-O-acetyl-3-phenylpropionate.

### EXAMPLES 2-5

Enzymatic transesterification for kinetic resolution of ethyl 3-hydroxy-3-phenylpropionate was carried out using the following agents as shown in Table 1 as acylating agents instead of vinyl acetate in the Example 1. Then, the conversion and enantiomeric excess are as follows.

TABLE 1

| | | Acylating | Reaction | | % ee for | % ee for |
|---|---|---|---|---|---|---|
| Example | lipase | agent | time(hr) | Conversion (%) | (S)-alcohol | (R)-ester |
| 2 | PS-C | Vinyl | 20 | 58.9 | 100 | 90.0 |
| | | propionate | | | | |
| 3 | PS-C | Isopropenyl | 20 | 64.3 | 100 | 66.2 |
| | | acetate | | | | |
| 4 | PS-C | Acetic | 20 | 46.7 | 91.5 | 80.7 |
| | | anhydride | | | | |
| 5 | PS-C | Butyric | 20 | 50.9 | 100 | 100 |
| | | anhydride | | | | |

### EXAMPLES 6-9

Enzymatic transesterification for kinetic resolution of ethyl 3-hydroxy-3-phenylpropionate was carried out using lipases as shown in Table 2 instead of lipase PS-C in the Example 1. The conversion and enantiomeric excess are as follows.

TABLE 2

| | Kind of | Reaction | Conversion | % ee for (S)- | % ee for |
|---|---|---|---|---|---|
| Example | lipase | Time(hr) | (%) | alcohol | (R)-ester |
| 6 | CRL | 188 | 41.5 | 20.3 | 32.8 |
| 7 | CAL | 42 | 54.1 | 100 | 95.8 |
| 8 | PS | 160 | 50.4 | 83.6 | 100 |
| 9 | PS-D | 20 | 50.9 | 100 | 100 |

### EXAMPLES 10

Enzymatic transesterification of ethyl 3-hydroxy-3-phenylpropionate was carried out using vinyl propionate as acylating agent and isopropylether as organic solvent instead of t-butylmethylether in the Example 8 for 160 hours. Then, the result is as follows; at 52.1% of conversion, 99.9. % ee for (S)— ethyl 3-hydroxy-3-phenylpropionate and 98.7% ee for (R)-ethyl 3-O-propionyl-3-phenylpropionate.

### EXAMPLES 11-12

Enzymatic transesterification of ethyl 3-hydroxy-3-phenylpropionate was carried out using the follow solvents as shown in Table 3 instead of t-butylmethylether in the Example 1. The conversion and enantiomeric excess are as follows.

TABLE 3

| | | | | % ee | |
|---|---|---|---|---|---|
| ex- | | | | for | |
| am- | | Reaction | Conversion | (S)- | % ee for |
| ple | Organic solvent | time(hr) | (%) | alcohol | (R)-ester |
| 11 | Tetrahydrofurane | 135 | 61.3 | 100 | 81.3 |
| 12 | Methylene | 135 | 61.3 | 100 | 86.6 |
| | chloride | | | | |

### EXAMPLES 13-14

The acylating agent (4.95 ml) as shown in Table 4 was placed iii a 15 ml vial without adding organic solvent. Then, ethyl 3-hydroxy-3-phenylpropionate (0.05 ml, 1% v/v) and lipase PS-C (0.2 g, 4% w/v) were added to the vial containing the acylating agent. The conversion and enantiomeric excess are as follows.

TABLE 4

| Ex- | Acylating | Reaction | Conversion | % ee for | % ee for |
|---|---|---|---|---|---|
| ample | agent | time(hr) | (%) | (S)-alcohol | (R)-ester |
| 13 | Vinyl | 20 | 50.2 | 96.1 | 93.5 |
| | acetate | | | | |
| 14 | Vinyl | 20 | 55.1 | 96.3 | 95.4 |
| | propionate | | | | |

### INDUSTRIAL APPLICABILITY

In accordance with this invention, the process is environmentally friendly and economical because lipases can be reused. With using a selected lipase and an acylating agent, (S)- or (R)-ethyl 3-hydroxy-3-phenylpropionate of high optical purity can be produced on the industrial scale.

What is claimed as:

1. A process for preparing optically active ethyl 3-hydroxy-3-phenylpropionate and their esters from racemic ethyl 3-hydroxy-3-phenylpropionate by enzyme-mediated esterification with acylating agent in organic solvent.

2. A process for preparing optically active ethyl 3-hydroxy-3-phenylpropionate and their esters from racemic ethyl 3-hydroxy-3-phenylpropionate by enzyme-mediated esterification with acylating agent only without using organic solvent.

3. The process according to claim 1 or 2, wherein said above acylating agents are chosen from vinyl esters, isopropenyl acetate and anhydride compounds.

4. The process according to claim 1, wherein said above organic solvents are chosen from isopropylether, t-butylmethylether, tetrahydrofurane and methylenechloride.
